(19) United States
(12) Patent Application Publication
(10) Pub. No.: US 2022/0047858 A1
(43) Pub. Date: Feb. 17, 2022

(54) MICRONEEDLE HAVING LAYERED STRUCTURE

(71) Applicant: FEROKA INC., Seoul (KR)

(72) Inventors: Induk LEE, Seoul (KR); Yeo Myung LIM, Seoul (KR); Yiseul JEON, Seoul (KR)

(21) Appl. No.: 17/276,774

(22) PCT Filed: Jan. 23, 2020
(86) PCT No.: PCT/KR2020/001201

§ 371 Date: Mar. 16, 2021

(30) Foreign Application Priority Data
Jan. 25, 2019 (KR) 10-2019-0010012
Jan. 25, 2019 (KR) 10-2019-0010013

(51) Int. Cl. A61M 37/00

(52) U.S. Cl. CPC A61M 37/0015; A61M 2037/0053; A61M 2037/0023

(57) ABSTRACT

The present invention relates to a tree-shaped microneedle having a layered structure with three or more layers and a manufacturing method therefor, and is a technique relating to a liquid injection-type microneedle or a nanobubble micro.

### TECHNICAL FIELD

The present invention relates to a micro-needle and a method for manufacturing the same, and more particularly, relates to a micro-needle having a tree-shaped structure of three layers or more, and a method for manufacturing the same.

### BACKGROUND ART

When a bioactive material is injected into skin of a person, an existing injection needle may be used, but the bioactive material may cause pains at an injection portion, damage to the skin, and infection due to the injection needle.

Accordingly, in recent years, methods for delivering the bioactive material to the interior of the skin by using a micro-needle (or an ultrafine needle) have been actively studied. The micro-needle may have a diameter of several tens or several hundreds of micrometers to penetrate a horny layer of the skin that is a main barrier layer.

Unlike the existing injection needle, the features of the micro-needle are penetration into skin with no pains and no injury. Further, the micro-needle may require a physical hardness of a degree because it has to penetrate the horny layer of the skin. Further, a proper length may be required so that the bioactive material reaches an epidermal layer or a dermic layer of the skin. Furthermore, in order that the bioactive material of several hundreds of micro-needles may be effectively delivered to the interior of skin, a predetermined period of time should be maintained until the micro-needles are solved after the micro-needles are inserted into the skin while increasing the rate of penetration of the micro-needles in the skin.

Existing methods for manufacturing the micro-needle may include may include a molding method and a tension method.

In the method for manufacturing a micro-needle by using the molding method, because the aspect ratio of the micro-needle is low due to the characteristics of the mold, it is difficult to punch the skin and the density of the micro-needles is low.

The method for manufacturing a micro-needle by using the tension method is a method for manufacturing the micro-needle by dropping a material on a patch, prolonging the patch, drying the patch, and cutting off a thinned portion of the patch, and due to the characteristics, the length of the micro-needle is not constant and a lot of pains may be felt due to the shape.

Furthermore, both of the molding method and the tension method require high costs, and thus act as an obstacle to the growth of the markets, and because micro-needles of a high density cannot be disposed, they should be attached for around 2 hours, which is inconvenient. Moreover, because it is difficult to increase the density of the micro-needles according to both the two methods, it is recommended that the micro-needles manufactured through the two methods be attached for two hours or more, but the time is longer as compared with a general patch that has to be attached for about 20 minutes.

The reason why the attachment time is long is that the density of the needles is low. Because the density of the micro-needles is low, the entire surface area of the micro-needles included in the patch is small, and because the contact area with the skin is small, the speed of reaction with the skin is inevitably low. However, because it is difficult to further increase the density of the micro-needles according to the two existing methods, the speed of the reaction with the skin cannot become higher.

When manufacturing of a medical micro-needle instead of a cosmetic purpose micro-needle is considered, the limits in the two existing methods become more distinct. According to both the two existing methods, it is necessary to manufacture the entire needle of a uniform mixture of the same concentration when the vaccine and the medicine are mixed. However, it is difficult to make the sizes of the needles constant, and because it is impossible to adjust the degree, by which the needles penetrate into skin, due to the medicine left on a border surface between the patch and the skin and in a passage, it is almost impossible to injection a proper amount of the medicine.

Accordingly, a need for a micro-needle having a multilayered structure started to be suggested, and for example, an assertion that a micro-needle having the multilayered structure is necessary when a proper amount of insulin is to be injected has been suggested (Ito et al., Diabetes Technology & Therapeutics, 2012, 14, 10).

### DETAILED DESCRIPTION OF THE INVENTION

### Technical Problem

The present invention suggests a micro-needle that may reinforce preservation of a medical fluid, facilitate penetration of the micro-needle into skin, and inject the medical fluid in a liquid state by manufacturing the liquid injection type micro-needle of a tree-shaped structure of three layers or more, including a middle part including the fusible medical liquid in a cavity, a lower end part that supports the middle part, and an upper end part located at an upper end of the middle part.

The present invention also suggests a micro-needle that may adjust a fusion speed, at which a medical fluid is fused, due to an increase of the surface area by nano-bubbles, by manufacturing a nano-bubble micro-needle of a tree-shaped structure of three layers or more, including an upper end part, a middle part, and a lower end part formed of a plurality of nano-bubbles.

### Technical Solution

According to an aspect of the present invention, there is provided a liquid injection type micro-needle having a structure of three layers or more, the liquid injection type micro-needle including a middle part penetrating into an interior of skin and including a fusible medical fluid in a cavity thereof, a lower end part supporting the middle part, and an upper end part located at an upper end of the middle part and configured to facilitate the penetration.

The middle part may include the cavity having a groove shape of a specific size in an interior thereof, and the medical fluid in a liquid state may be contained in the cavity.

The middle part may seal the medical fluid by blocking an upper end of the cavity, in which the medical fluid is contained.

A surface of the cavity, which contacts the medical fluid, may be coated with a waterproof material, which does not react with the medical fluid.

The upper end part and the middle part may have a pyramid or conical shape, and the lower end part may have a prism or cylindrical shape.

The lower end part may include a fusible material connecting a base and the liquid injection type micro-needle, and may separate the liquid injection type micro-needle from the base.

The upper end part, the middle part, and the lower end part may be formed of different materials.

According to another aspect of the present invention, there is provided a method for manufacturing a liquid injection type micro-needle, the method including forming a lower end part, forming an initial middle part of a shape of a cavity, on the lower end part, injecting a medical fluid after the liquid injection type micro-needle penetrates into skin, forming the middle part by blocking an upper end of the cavity, into which the medical fluid is injected, and forming an upper end part on the middle part.

According to another aspect of the present invention, there is provided a nano-bubble micro-needle having a structure of three layers or more, the nano-bubble micro-needle including a middle part formed of a compound penetrating an interior of skin and including a medicine component, a lower end part supporting the middle part and formed of a plurality of nano-bubbles, and an upper end part located at an upper end of the middle part and configured to facilitate the penetration.

The upper end part and the middle part may have a pyramid or conical shape, and the lower end part may have a prism or cylindrical shape.

The upper end part, the middle part, and the lower end part may be formed of different materials.

The lower end part may include the plurality of nano-bubbles in an interior of the prism or cylindrical shape.

Sizes and the amount of the nano-bubbles may be adjusted according to a depth degree, a fusion speed, and a kind of a material of the lower end part penetrating into the skin.

The lower end part may be formed of a fusible material connecting a base and the nano-bubble micro-needle, and may separate the nano-bubble micro-needle from the base.

According to another aspect of the present invention, there is provided a method for manufacturing a nano-bubble micro-needle, the method including forming a lower end part formed of a plurality of nano-bubbles, forming a middle part penetrating into an interior of skin and formed of a compound including a medical component on the lower end part, and forming an upper end part on the middle part.

### Advantageous Effects of the Invention

According to the embodiment of the present invention, the preservation of the medical liquid may be reinforced, the penetration of the liquid injection type micro-needle may be facilitated, and the medical liquid in the liquid state may be injected, by manufacturing the liquid injection type micro-needle of the structure of three layers or more.

Furthermore, according to the embodiment of the present invention, the weight of the nano-bubble micro-needle may be made light, the speed, at which the medical liquid is fused, may be increased due to the increase in the surface area due to the nano-bubbles, and the strength of the nano-bubble micro-needle may be maintained, by manufacturing the nano-bubble micro-needle having the structure of three layers or more, including the lower end part formed of the plurality of nano-bubbles. Moreover, according to the embodiment of the present invention, the nano-bubble micro-needle of the structure of three layers or more may adjust the speed, at which the medical fluid is fused in the interior of the skin according to the sizes and the amount of the nano-bubbles.

Furthermore, according to the embodiment of the present invention, the preservation of the medical fluid may be reinforced and the penetration of the nano-bubble micro-needle into the skin may be made easy, by manufacturing the nano-bubble micro-needle of the structure of three layers or more.

In addition, according to the embodiment of the present invention, the liquid injection type micro-needle or the nano-bubble micro-needle of the structure of three layers or more, which is manufactured by using the 3D printing technology, is advantageous in the technical and economic aspects of the penetration of the micro-needle into the skin, presence of pains, the density of needles, the attachment time, the precision, the costs, and the expandability as compared with the existing methods.

Furthermore, when the liquid injection type micro-needle or the nano-bubble micro-needle is manufactured according to the present invention, a high competitiveness may be secured in the wrinkle improvement cosmetics markets and the medical markets.

That is, according to the present invention, because the liquid injection type micro-needle or the nano-bubble micro-needle having the structure of three layers or more, including the upper end part, the middle part, and the lower end part of different forms, it is suitable for a medical purpose.

### DESCRIPTION OF THE DRAWINGS

FIG. 1 illustrates a perspective view of a liquid injection type micro-needle according to an embodiment of the present invention;

FIGS. 2A and 2B illustrate cross-sectional views of a liquid injection type micro-needle including a cavity and a medical fluid according to an embodiment of the present invention;

FIG. 3 illustrates a cross-sectional view of a liquid injection type micro-needle of a structure of three layers or more according to an embodiment of the present invention;

FIG. 4 illustrates exemplary views for comparing micro-needles manufactured according to an existing method and a method of the present invention;

FIG. 5 illustrates a perspective view of a liquid injection type micro-needle patch manufactured according to an embodiment of the present invention;

FIG. 6 illustrating a flowchart of operations of a method for manufacturing a liquid injection type micro-needle according to an embodiment of the present invention;

FIG. 7 illustrates operations of manufacturing a liquid injection type micro-needle through a method for manufacturing a liquid injection type micro-needle according to an embodiment of the present invention;

FIG. 8 illustrates a perspective view of a nano-bubble micro-needle according to an embodiment of the present invention;

FIGS. 9A and 9B illustrate cross-sectional views of a micro-needle formed of nano-bubbles according to an embodiment of the present invention;

FIGS. 10A and 10B illustrate cross-sectional views for explaining a structural feature of a nano-bubble micro-needle formed according to an embodiment of the present invention;

FIG. 11 illustrates exemplary views for comparing micro-needles manufactured according to an existing method and a method of the present invention;

FIG. 12 illustrates a perspective view of a nano-bubble micro-needle patch manufactured according to an embodiment of the present invention;

FIG. 13 illustrating a flowchart of operations of a method for manufacturing a nano-bubble micro-needle according to an embodiment of the present invention; and

FIG. 14 illustrates operations of manufacturing a micro-needle through a nano-bubble method for manufacturing a nano-bubble micro-needle according to an embodiment of the present invention.

### BEST MODE

Hereinafter, embodiments of the present invention will be described in detail with reference to the accompanying drawings. However, the present invention is neither limited nor restricted by the embodiments. Furthermore, the same reference numerals in the drawings denote the same members.

Furthermore, the terminologies used herein are used to properly express the embodiments of the present invention, and may be changed according to the intentions of the user or the manager or the custom in the field to which the present invention pertains. Therefore, definition of the terms should be made according to the overall disclosure set forth herein.

The essence of the embodiments of the present invention is that, by manufacturing a liquid injection type micro-needle of a structure of three layers or more including a middle part including a medical fluid, an upper part located at an upper end of the middle part to facilitate penetration of the medical fluid into skin, and a lower end part that supports the middle part, the preservation of the medical fluid may be reinforced, the penetration of the medical fluid into the skin may be facilitated, and the medical fluid may be injected in a liquid state. Then, the liquid injection type micro-needle according to the embodiment of the present invention is characterized in that it has a structure of three layers or more.

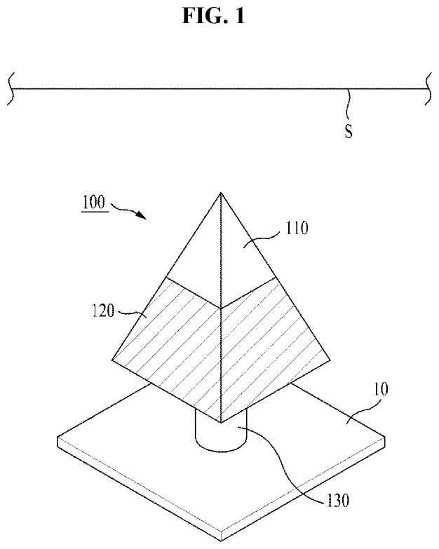

Hereinafter, the embodiments of the present invention will be described in detail with reference to FIGS. 1 to 7.

FIG. 1 illustrates a perspective view of a liquid injection type micro-needle according to an embodiment of the present invention.

Referring to FIG. 1, the liquid injection type micro-needle 100 according to the embodiment includes an upper end part 110, a middle part 120, and a lower end part 130.

The upper end part 110 is located at an upper end of the middle part 120 and facilitates penetration of the liquid injection type micro-needle 100 into skin “S”. A tip end of the upper end part 110 has a sharp end shape, for example, a pyramid shape such as a triangular pyramid shape, a rectangular pyramid shape, a pentagonal pyramid shape, or a hexagonal pyramid shape, with respect to the penetration direction, in which the liquid injection type micro-needle 100 penetrates into the skin “S”, and for example, may facilitate the penetration of the liquid injection type micro-needle 100 into the skin “S”. Then, in order to facilitate punching of the skin “S”, the upper end part 110 is formed of a material having a strength that is higher than those of the middle part 120 and the lower end part 130.

The upper end part 110 according to the embodiment of the present invention may facilitate the penetration of the liquid injection type micro-needle 100 into the skin “S”, and may protect the middle part 120 including a medical fluid.

According to the embodiment, the upper end part 110 may be formed of a water-soluble material that penetrates into the skin “S” and is fused. For example, the water-soluble material may be at least any one of trehalose, oligosaccharide, sucrose, maltose, lactose, cellobiose, hyaluronic acid, alginic acid, pectin, carrageenan, chondroitin sulfate, dextran sulfate, chitosan, poly lysine, collagen, gelatin, carboxy methyl chitin, fibrin, agarose, poly vinyl pirrolidone (PVP), poly ethylene glycol (PEG), poly meta crylate, hydroxyl propyl methyl cellulose (HPMC), ethylene cellulose (EC), hydroxyl propyl cellulose (HPC), carboxy methyl cellulose, cyclodextrin, and gentiobiose.<0}

The middle part 120 may penetrate into the skin “S” through the upper end part 110 and includes the fusible medical fluid in a cavity. When the middle part 120 penetrates into the skin “S” through the upper end part 110, the medical fluid that is a fusible water-soluble polymer included in the cavity may be absorbed into the interior of the skin “S”.

The middle part 120 shows a shape of a pyramid such as a triangular pyramid, a rectangular pyramid, a pentagonal pyramid, or a hexagonal pyramid or a cone, from which the upper end part 110 is removed, and may include a cavity area that may contain the medical fluid in the interior thereof. Then, it may be preferable that the cavity area is located in an upper end area that is above the center of the middle part 120, but according to embodiments, the location, the size, and the form of the cavity area may be variously applied according to a time point, at which the medical fluid is introduced, an injection time, and a dose of the medical fluid. Moreover, the size and the location of the cavity may be adjusted according to the amount, the vaporization speed, and the temperature of the medical fluid, the form of the middle part 120 for manufacturing the liquid injection type micro-needle 100, the viscosity of the medical fluid, the concentration of the medical fluid, a solvent used, and the thickness of the covering part that covers an upper end of the cavity.

The middle part 120 may be formed of a water-soluble material like the upper end part 110 that penetrates into the skin “S”. Meanwhile, because the middle part 120 includes the cavity, and the medical fluid in a liquid state that is contained in the cavity, it is preferable that, among water-soluble materials, a material that is different from that of the upper end part 110 be used in the middle part. Because the medical fluid in the liquid state or in the liquid state that may be solidified, which is contained in the cavity area, may be absorbed by the material of the middle part 120 when the medical fluid is injected into the middle part 120, it is preferable that the middle part 120 is formed of a water-soluble material that is different from that of the upper end part 110, and a surface of the cavity, in which the medical fluid is contained, is coated of a waterproof material.

The medical fluid contained in the cavity in the middle part 120 may be formed of a bio-compatible material and an additive. For example, the bio-compatible material may include at least any one of carboxy methyl cellulose (CMC), hyaluronic acid (HA), alginic acid, pectin, carrageenan, chondroitin sulfate, dextran sulfate, chitosan, poly lysine, carboxy methyl chitin, fibrin, agarose, pullulan, polyan hydride), poly ortho ester, poly ether ester, poly ester amide, poly butyric acid, poly valeric acid), poly acrylate, an ethylene-vinyl acetate polymer, acryl replaced cellulose acetate, poly vinyl chloride, poly vinyl fluoride, poly vinyl imidazole, chloro sulphonate poly olefins, poly ethylene oxide, poly vinyl pirrolidone (PVP), hydroxyl propyl methyl cellulose (HPMC), ethylene cellulosed (EC), hydroxyl propyl cellulose (HPC), carboxy methyl cellulose, cyclodextrin, maltose, lactose, trehalose, cellobiose, isomaltose, turanose, and lactulose, or may include at least any one of a copolymer of monomers forming polymers, and cellulose.

Furthermore, the additive may include at least any one of trehalose, oligosaccharide, sucrose, maltose, lactose, cellobiose, hyaluronic acid, alginic acid, pectin, carrageenan, chondroitin sulfate, dextran sulfate, chitosan, poly lysine, collagen, gelatin, carboxy methyl chitin, fibrin, agarose, poly vinyl pirrolidone (PVP), poly ethylene glycol (PEG), poly meta crylate, hydroxyl propyl methyl cellulose (HPMC), ethylene cellulose (EC), hydroxyl propyl cellulose (HPC), carboxy methyl cellulose, cyclodextrin, gentiobiose, alkyl trimethyl ammonium bromide (cetrimide), hexa decyltrimethyl ammonium bromide (CTAB), gentian violet, benzethonium chloride, docusate sodium salt, a SPAN-type surfactant), polysorbate (Tween), sodium dodecyl sulfate (SDS), benzalkonium chloride, and glyceryl oleate.

The medical fluid contained in the cavity in the middle part 120 may be formed by mixing a bio-compatible material and an effective component. The effective component includes, without being limited thereto, a protein/peptide medicine, and includes at least one of a hormone, a hormone agonist, an enzyme, an enzyme inhibitor, a signal transduction protein or a portion thereof, an antibody and a portions thereof, a single chain antibody, a binding protein or a binding domain thereof, an antigen, an adherence protein, a structural protein, a regulatory protein, a toxo protein, cytokine, a transcriptional regulatory factor, a blood coagulation factor, and a vaccine. In more detail, the protein/peptide medicine may include any one of insulin, insulinlikegrowth factor 1 (IGF-1), a growth hormone, erithropoietin, granulocyte-colony stimulating factors (G-CSFs), granulocyte/macrophage-colony stimulating factors (GM-CSFs), interferon-alpha, interferon-beta, interferon-gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRHII), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormonereleasing hormone (LHRH), nafarelin, parathyroid hormone, pramlintide, enfuvirtide (T-20), thymalfasin, and ziconotide.

The solvent of the medical fluid contained in the cavity in the middle part 120 may solve the bio-compatible material. The solvent may include at least any one of inorganic solvents and organic solvents including DI water, methanol, ethanol, chloroform dibutyl phthalate, dimethyl phthalate, ethyl lactate, glycerin, isopropyl alcohol, lactic acid, and propylene glycol.

According to the liquid injection type micro-needle 100 according to the embodiment of the present invention, a proper amount of the medical fluid is injected by forming the cavity of a specific area in the interior of the middle part 120 and injecting the medical fluid in the liquid state, which is contained in the interior of the cavity, into the interior of the skin “S”, and accordingly, the present invention may reinforce the preservation of the medical fluid, facilitate the penetration of the liquid injection type micro-needle 100 into the interior of the skin, and allow the medical fluid in the liquid state to be injected.

The lower end part 130 supports the middle part 120. The lower end part 130 has a prism shape, such as a triangular prism, a rectangular prism, a pentagonal prism, and a hexagonal prism or a cylindrical shape, and supports the upper end part 110 and the middle part 120.

The lower end part 130 may have a diameter and a height of a specific size, and may indicate the depth degree, by which the liquid injection type micro-needle 100 penetrates into the interior of the skin “S”. For example, the depth degree, by which the upper end part 110 and the middle part 120 including the medical fluid penetrate into the interior of the skin “S”, may be estimated according to the diameter and the height of the lower end part 130, and the height of the lower end part 130 may be adjusted according to the depth degree, by which the medical fluid penetrates, based on the kind of the medical fluid, the state of the medical fluid, a time point, at which the medical fluid is injected, an injection time, and a dose. Furthermore, the diameter of the lower end part 130 may be adjusted according to the weights and the sizes of the upper end part 110 and the middle part 120, a degree, by which the medical fluid is supported, and a time period, for which the lower end part 130 is fused in the interior of the skin “S”.

The lower end part 130 is formed of a fusible material that connects the base 10 and the liquid injection type micro-needle 100, and separates the liquid injection type micro-needle 100 from the base 10. For example, the lower end part 130 may be formed of a water-soluble fusible material and be rapidly fused, and accordingly, the micro-needle 100 formed on the base 10 may be rapidly separated.

Then, the lower end part 130 may be formed of a water-soluble material like the upper end part 110 and the middle part 120 that penetrate into the skin “S”. However, the lower end part 130 may be formed of, among the soluble materials, a material that is fused more rapidly than the upper end part 110 and the middle part 120. Because the upper end part 110 is for facilitating the punching of the skin more easily, the middle part 120 includes the medical fluid in the liquid state and is for more efficient injection, and the lower end part 130 is for rapider separation of the micro-needle 100 formed on the base 10 and the depth degree of the liquid injection type micro-needle 100 in to the interior of the skin “S”, the micro-needle 100 according to the embodiment of the present invention includes the upper end part 110, the middle part 120, and the lower end part 130 of a structure of three layers or more formed of the different materials.

Because the lower end part 130 according to the embodiment of the present invention functions to support the upper end part 110 and the middle part 120 in the liquid injection type micro-needle 100, it may indicate the depth degree, by which the liquid injection type micro-needle 100 penetrates into the skin. As illustrated in FIG. 1, the lower end part 130 has a prism or cylindrical shape and occupies a size and a volume that are smaller than those of the upper end part 110 and the middle part 120, and accordingly, the lower end part 130 shows an effect of minimizing the area, the volume, and the weight of the liquid injection type micro-needle 100, and supporting the liquid injection type micro-needle 100 such that a proper amount of the medical fluid is injected due to the shape of the proper size, the proper height, and the proper diameter according to the depth degree, by which the liquid injection type micro-needle 100 penetrates into the skin “S”.

As illustrated in FIG. 1, the liquid injection type micro-needle 100 may be formed on the base 10. The base 10 is not provided with the medical fluid, and may be separated after the liquid injection type micro-needle 100 of the upper end part 110, the middle part 120, and the lower end part 130 penetrate into the skin “S”. For example, the base 10 is provided in the same form as a kind of a patch, and may be adhered to the skin “S”.

Unlike the liquid injection type micro-needle 100 that penetrates into the skin “S”, the base 10 may be formed of a non-aqueous material that is not fused. Accordingly, because the base 10 does not interfere with the penetration force of the liquid injection type micro-needle 100, it may guide supply of the proper amount of the medical fluid included in the middle part 120.

For example, the base 10 may be formed of at least any one of a group including poly ethylene (PE), poly propylene (PP), poly tetra fluoro ethylene PTFE, poly methyl meta acrylate (PMMA), ethylene vinyl acetate (EVA), poly capro lactone (PCL), poly urethane (PU), poly ethylene terephthalate (PET), poly ethylene grlycol (PEG), poly vinyl alcohol (PVA), poly lactide (PLA), a poly lactide-glycoride (PLGA) copolymer, and poly glycoric acid.

As illustrated in FIG. 1, the liquid injection type micro-needle 100 according to the embodiment of the present invention may reinforce preservation of the medical fluid, facilitate penetration of the liquid injection type micro-needle 100 into the skin, and may inject a proper amount of the medical fluid in the liquid state, by forming the middle part 120 including the medical fluid, the upper end part 110 located at the upper end of the middle part 120 to facilitate the penetration of the liquid injection type micro-needle 100 into the skin S″, the lower end part 130 that supports the middle part 120 such that they have a structure of a tree shape of three layers or more.

Furthermore, the liquid injection type micro-needle 100 according to the embodiment of the present invention has a structure of a tree shape of three layers or more, and thus may increase the penetration rate (60% or more) of the structure and the absorption rate of the available substance in the skin, by minimizing the penetration resistance due to the elasticity of the skin when the liquid injection type micro-needle 100 is attached to the skin. Furthermore, the liquid injection type micro-needle 100 of the tree shape can easily penetrate into the skin by applying the structure of three layers or more to maximize the mechanical strength of the structure.

Furthermore, the upper end part 110 of a pyramid or conical shape, the middle part 120 of a pyramid or conical shape, and the lower end part 130 of a prism or cylindrical shape, which form the liquid injection type micro-needle 100 according to the embodiment of the present invention are manufactured through a 3D printing technology. Because the present invention uses a 3D printing method, attachment time may be made very short, precision may also be made high, the price may be made low, the density of the liquid injection type micro-needle 100 in the micro-patch may be increased, and the aspect ratio may be improved as compared with the existing method.

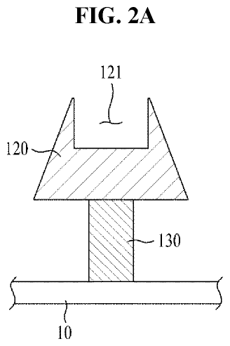

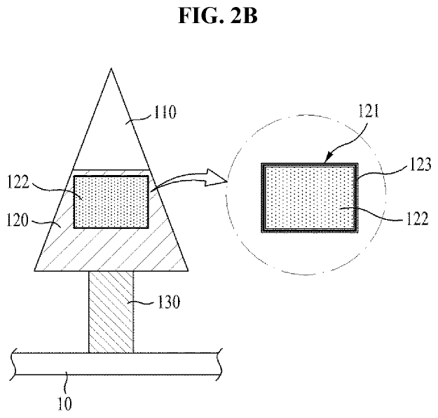

FIGS. 2A and 2B illustrate cross-sectional views of a liquid injection type micro-needle including a cavity and a medical fluid according to an embodiment of the present invention.

Referring to FIG. 2A, the liquid injection type micro-needle 100 according the embodiment of the present invention includes the middle part 120 including the cavity 121. The cavity 121 may have a groove shape in the middle part 120, and may have a form and a size for including the medical fluid.

Referring to FIG. 2B, the liquid injection type micro-needle 100 according to the embodiment of the present invention may include the cavity 121 containing the medical fluid 122. Then, the entire cavity 121 containing the medical fluid 122 may be located in the interior of the middle part 120, and when the medical fluid 122 is injected into the area of the cavity 121 in FIG. 2A, the upper end of the cavity is blocked to seal the medical fluid 122. Thereafter, the liquid injection type micro-needle 100 according to the embodiment of the present invention is manufactured by forming the upper end part 110 on the middle part 120.

As illustrated in FIG. 2B, a surface 123 of the cavity in contact with the medical fluid 122 may be coated with a waterproof material. The medical fluid 122 according to the embodiment of the present invention may be in a liquid state or in a liquid state that may be solidified. Because the medical fluid 122 in the liquid state may be absorbed by the middle part 120, the surface 123 of the cavity is coated with the waterproof material to block the medical fluid 122.

For example, the surface 123 of the cavity may be coated with the waterproof material including a mineral-based material or a lipid-based material. Here, the waterproof material may include at least one of beeswax, oleicacid, soy fatty acid, castor oil, phosphatidylcholine, d-α-tocopherol/Vitamin E, corn oil, corn oil mono-ditridiglycerides, cottonseed oil, olive oil, peanut oil, peppermint oil, safflower oil, sesame oil, soybean oil, hydrogenated vegetable oils, hydrogenated soybean oil, caprylic/capric triglycerides derived from coconut oil or palm see oil, and phosphatidylcholine, or a mixture thereof.

According to the embodiment, the surface 123 of the cavity may be coated with different waterproof materials according to the kind and the state of the medical fluid 122 injected into the cavity, and the size, the height, and the form of the cavity 121 in the middle part 120 may be determined according to the kind of the medical fluid 122, the state of the medical fluid 122, the time point, at which the medical fluid 122 is injected, and the injection time period, and the dose.

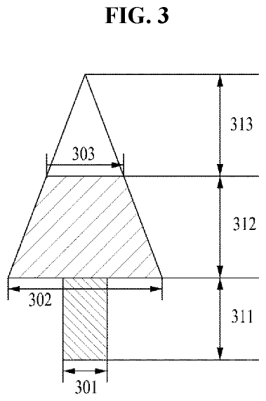

FIG. 3 illustrates a cross-sectional view of a liquid injection type micro-needle of a structure of three layers or more according to an embodiment of the present invention.

The liquid injection type micro-needle 100 according to the embodiment of the present invention is a micro-structure having three layers or more, and includes the upper end part 110 and the middle part 120 of a pyramid or conical shape, and the lower end part 130 of a prism or cylindrical shape.

As illustrated in FIG. 3, the diameter 302 of the bottom surface of the middle part is larger than the diameter 303 of the bottom surface of the upper end part or the diameter 301 of the bottom surface of the lower end part, and the diameter 303 of the bottom surface of the upper end part is larger than the diameter 301 of the bottom surface of the lower end part. Their sizes may be determined in the sequence of the diameter 302 of the bottom surface of the middle part, the diameter 303 of the bottom surface of the upper end part, and the diameter 301 of the bottom surface of the lower end part.

Furthermore, the height 312 of the middle part may be larger than the height 313 of the upper end part, and the sum of the height 312 of the middle part and the height 313 of the upper end part may be larger or smaller than the height 311 of the lower end part. That is, in the liquid injection type micro-needle 100 according to the embodiment of the present invention, the height 312 of the middle part may be largest, and the height 313 of the upper end part and the height 311 of the lower end part may be the same or may vary according to the embodiment, to which the liquid injection type micro-needle 100 according to the embodiment of the present invention is applied. However, the height 311 of the lower end part, the height 312 of the middle part, and the height 313 of the upper end part of the liquid injection type micro-needle 100 according to the embodiment of the present invention are not limited to those illustrated in FIG. 3, and may be various heights according to the embodiment applied.

Because the middle part 120 of the liquid injection type micro-needle according to the embodiment of the present invention has the cavity containing the medical fluid, it may have the widest volume, the largest bottom surface diameter 302, and the largest height 312. Because the upper end part 110 has a pyramid or conical shape for penetrating into the skin “S”, the diameter 303 of the bottom surface of the upper end part is the same as the diameter of the upper surface (or the tip end) of the middle part, and may be determined according to the cross-sectional area of the pyramid or conical shape that forms the middle part 120. Furthermore, the height 313 of the upper end part may be determined according to the pyramid or conical shape of the middle part.

Because the lower end part 130 of the liquid injection type micro-needle according to the embodiment of the present invention functions to support the upper end part 110 and the middle part 120 in the liquid injection type micro-needle 100, it may indicate the depth degree, by which the liquid injection type micro-needle 100 penetrates. Accordingly, the lower end part 130 has a volume and a bottom surface diameter 301 that are smaller than those of the upper end part 110 and the middle part 120. However, the height 311 of the lower end part may be determined according to the depth degree, by which the liquid injection type micro-needle penetrates into the skin.

The lower end part 130 has a prism or cylindrical shape and includes the bottom surface diameter 301 that is smaller than the diameter 303 of the bottom surface of the upper end part and the diameter 302 of the bottom surface of the middle part, and the volume of the lower end part 130 is also smaller than those of the upper end part 110 and the middle part 120. The lower end part 130 is adapted to indicate the depth degree, by which the liquid injection type micro-needle 100 penetrates into the skin “S” and support the upper end part 110 and the middle part 120, and thus minimizes the area, the volume, and the weight of the liquid injection type micro-needle 100 according to the embodiment of the present invention. Accordingly, the lower end part 130 shows an effect of supporting the liquid injection type micro-needle 100 that penetrates into the interior of the skin “S” such that a proper amount of the medical fluid may be injected due to a proper size, a proper height, and a proper diameter shape according to the depth degree of the liquid injection type micro-needle 100.

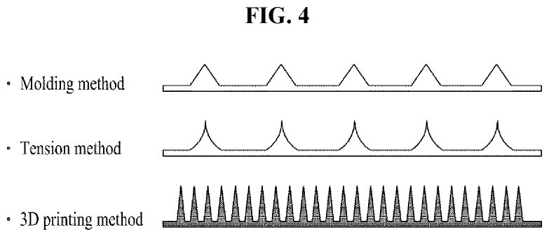

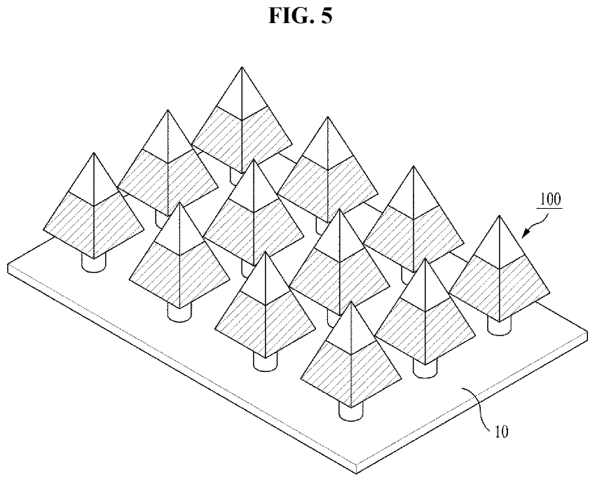

FIG. 4 illustrates exemplary views for comparing micro-needles manufactured according to an existing method and a method of the present invention. FIG. 5 illustrates a perspective view of a liquid injection type micro-needle patch manufactured according to an embodiment of the present invention.

Referring to FIG. 4, it can be seen that the density of the micro-needles is low in the molding method and the tension method whereas the density of the liquid injection type micro-needles according to the embodiment of the present invention manufactured by using a stacking method, for example, a 3D printing method is very high as compared with the existing methods due to the limitation of the molding method and the tension method, and it can be seen that the aspect ratio of the liquid injection type micro needles manufactured by the method according to the embodiment of the present invention is also higher than those of the molding method and the tension method is very high. Of course, the aspect ratio of the liquid injection type micro-needle may be adjusted in the method according to the present invention, and the aspect ratio may be determined according to a field, in which the liquid injection type micro-needle of the present invention is used, for example, a treatment or medical field.

According to the method (the 3D printing method) according to the present invention, it is advantageous to punch skin, there is no pain, and the density of the liquid injection type micro needles is higher than those of the molding method and the tension method. Furthermore, it can be seen that attachment time is very short as compared with the existing method in the method according to the present invention, it can be seen that precision is also high, and it can be seen that manufacturing costs are low and thus expansion performance is high as it uses a stacking method, for example, the 3D printing method. In this way, the method according to the present invention is very advantageous in the technical aspect and the economical aspect as compared with the existing methods, such as the molding method and the tension method.

That is, in the liquid injection type micro-needle realized by the stacking technology through the method according to the present invention, skin may be punched well and pains are very low due to the high aspect ratio and the attachment time is very short due to the high density of the liquid injection type micro-needles. In addition, the present invention may realize the liquid injection type micro-needle at a high precision of about 5 micrometers and a desired medicine may be disposed at a desired location, and thus the expansion performance is high.

The liquid injection type micro-needle 100 manufactured as described above, as illustrated in FIG. 5, may be manufactured by a plurality of liquid injection type micro-needle patches formed on the base 10, and may be easily applied to the medical field. That is, the present invention may secure a high competition in the medical market field by manufacturing the liquid injection type micro-needle 100 of the structure of three layers or more in the stacking method using the 3D printing.

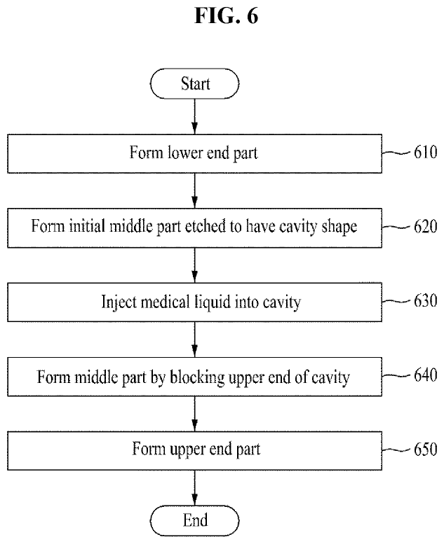

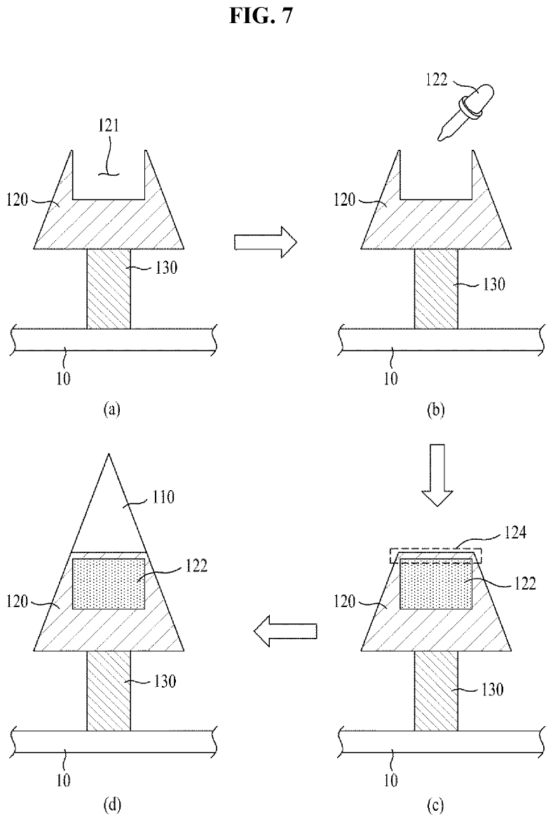

FIG. 6 illustrates a flowchart of operations of a method for manufacturing a liquid injection type micro-needle according to an embodiment of the present invention. FIG. 7 illustrates operations of manufacturing a liquid injection type micro-needle through a method for manufacturing a liquid injection type micro-needle according to an embodiment of the present invention.

The liquid injection type micro-needle 100 according to the present invention illustrated in FIG. 7, which is manufactured through the manufacturing method of FIG. 6 is manufactured through the 3D printing method.

Referring to FIG. 6, in operation 610, the lower end part is formed. In the method for manufacturing the liquid injection type micro-needle according to the embodiment of the present invention, the lower end part 130 having the prism or cylindrical shape may be formed on the base 10.

The lower end part 130 may have a diameter and a height of a specific size, and may indicate the depth degree, by which the liquid injection type micro-needle 100 penetrates into the interior of the skin “S”. For example, the depth degree, by which the upper end part 110 and the middle part 120 including the medical fluid penetrate into the interior of the skin “S”, may be estimated according to the diameter and the height of the lower end part 130, and the height of the lower end part 130 may be adjusted according to the depth degree, by which the medical fluid penetrates, based on the kind of the medical fluid, the state of the medical fluid, a time point, at which the medical fluid is injected, an injection time, and a dose. Furthermore, the diameter of the lower end part 130 may be adjusted according to the weights and the sizes of the upper end part 110 and the middle part 120, a degree, by which the medical fluid is supported, and a time period, for which the lower end part 130 is fused in the interior of the skin “S”.

In operation 620, the initial middle part having the shape of the cavity 121 is formed on the lower end part. As illustrated in FIG. 7A, in the method for manufacturing the liquid injection type micro-needle according to the embodiment of the present invention, the initial middle part having the shape of the cavity 121 is formed on the lower end part 130, and the upper end 124 of the cavity is opened. Then, it may be preferable that the cavity area is located in an upper end area that is above the center of the middle part 120, but according to embodiments, the location, the size, and the form of the cavity area may be variously applied according to a time point, at which the medical fluid is introduced, an injection time, and a dose of the medical fluid. Furthermore, the initial middle part may show the pyramid or conical shape including the shape of the cavity 121.

According to the embodiment, in operation 620, the method for manufacturing the liquid injection type micro-needle according to the embodiment of the present invention is a method, in which the initial middle part having the shape of the cavity 121 is manufactured in the 3D printing method and the manufactured initial middle part is stacked on the lower end part 130 or a method for manufacturing the initial middle part having the pyramid or conical shape including the shape of the cavity 121 on the lower end part 130, and may be illustrated as in FIG. 7A.

In operation 630, the fusible medical fluid 122 in the cavity 121 penetrates into the interior of the skin and is injected. Referring to FIG. 7B, in the method for manufacturing the liquid injection type micro-needle according to the embodiment of the present invention, the medical fluid 122 may be injected into the cavity 121. However, because the medical fluid 122 in the liquid state or the state of a liquid that may be solidified may be absorbed into the material of the middle part 120 when it is injected into the cavity 121, the surface of the cavity is coated with the waterproof material.

In operation 640, the middle part 120 is formed by blocking the upper end 124 of the cavity, into which the medical fluid 122 is injected. Referring to FIG. 7C, in the method for manufacturing the liquid injection type micro-needle according to the embodiment of the present invention, the cavity 121 including the medical fluid 122 is sealed in the middle part 121 by blocking the upper end 124 of the opened cavity when the medical fluid 122 is injected into the interior of the cavity 121. Then, in the method for manufacturing the liquid injection type micro-needle according to the embodiment of the present invention, the upper end 124 of the cavity may be blocked by the material of the middle part 120 through the 3D printing method.

Thereafter, in operation 650, the upper end part 110 is formed on the middle part 120. Referring to FIG. 7D, in the method for manufacturing the liquid injection type micro-needle according to the embodiment of the present invention, the upper end part 110 that facilitates the penetration of the liquid injection type micro-needle into the skin “S” may be formed to be located at the upper end of the middle part 120. A tip end of the upper end part 110 has a sharp end shape with respect to the penetration direction, in which the liquid injection type micro-needle 100 penetrates into the skin “S”, and may have, for example, a pyramid or conical shape to facilitate the penetration of the liquid injection type micro-needle 100 into the skin “S”.

The upper end part 110, the middle part 120, and the lower end part 130 of the liquid injection type micro-needle 100 according to the embodiment of the present invention may be formed of different materials. The upper end part 110, the middle part 120, and the lower end part 130 may be formed of the same water-soluble material, but the upper end part 110 that facilitates the penetration, the middle part 120 including the medical fluid, and the lower end part 130 that supports the middle part 120 and facilitates the separation from the base 10 may be formed of materials of different characters as long as they are soluble materials. For example, the upper end part 110 and the lower end part 130 may be materials that are fused in a shorter time than the middle part 120 thereof such that the proper amount of the medical fluid provided in the middle part 120 may be injected.

In the embodiments of the present invention, the preservation of the medicine is reinforced, the penetration of the nano-bubble micro-needle may be facilitated, the weight of the nano-bubble micro-needle may be made light, the fusion speed may be increased as the surface area increases due to the nano-bubbles, and strength may be maintained by manufacturing the nano-bubble micro-needle of a structure of three layers or more, including the middle part formed of a compound including a medicine component, the upper end part located at the upper end of the middle part to facilitate the penetration of the nano-bubble micro-needle into the interior of the skin, and the lower end part that supports the middle part and is formed of a plurality of nano bubbles. Then, the nano-bubble micro-needle according to the embodiment of the present invention is characterized in that it has a structure of three layers or more.

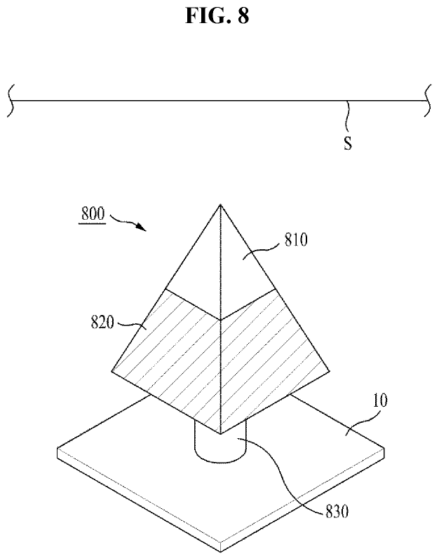

Hereinafter, the embodiments of the present invention will be described in detail with reference to FIGS. 8 to 14.

FIG. 8 illustrates a perspective view of a nano-bubble micro-needle according to an embodiment of the present invention.

Referring to FIG. 8, the nano-bubble micro-needle 800 according to the embodiment of the present invention includes an upper end part 810, a middle part 820, and a lower end part 830.

The upper part 810 is located at an upper end of the middle part 820 and facilitates penetration of the nano-bubble micro-needle 800. A tip end of the upper end part 810 has a sharp end shape such as a triangular pyramid shape, a rectangular pyramid shape, a pentagonal pyramid shape, or a hexagonal pyramid shape, with respect to the penetration direction, in which the nano-bubble micro-needle 100 penetrates into the skin “S”, and for example, may facilitate the penetration of the nano-bubble micro-needle 100 into the skin “S”. Then, in order to facilitate punching of the skin “S”, the upper end part 810 is formed of a material having a strength that is higher than those of the middle part 820 and the lower end part 830.

The upper end part 810 according to the embodiment of the present invention may facilitate the penetration of the nano-bubble micro-needle 800 into the skin “S”, and may protect the middle part 820 formed of a compound including a medicine component.

According to the embodiment, the upper end part 810 may be formed of a water-soluble material that penetrates into the skin “S” and is fused. For example, the water-soluble material may be at least any one of trehalose, oligosaccharide, sucrose, maltose, lactose, cellobiose, hyaluronic acid, alginic acid, pectin, carrageenan, chondroitin sulfate, dextran sulfate, chitosan, poly lysine, collagen, gelatin, carboxy methyl chitin, fibrin, agarose, poly vinyl pirrolidone (PVP), poly ethylene glycol (PEG), poly meta crylate, hydroxyl propyl methyl cellulose (HPMC), ethylene cellulose (EC), hydroxyl propyl cellulose (HPC), carboxy methyl cellulose, cyclodextrin, and gentiobiose.

The middle part 820 may penetrate into the skin “S” through the upper end part 810 and is formed of a compound including a medicine component. The middle part 820 is formed of a compound including the medicine component, and is solidified. Accordingly, when the middle part 820 penetrates into the skin “S” through the upper end part 810, the solidified medicine component may be fused and be absorbed into the skin “S”.

The middle part 820 of the nano-bubble micro-needle 800 according to the embodiment of the present invention is formed of a compound including a medicine component, that is, solidified, but may have a form including the cavity that may include the medicine in the liquid state according to the embodiment.

The middle part 820 shows a shape of a pyramid such as a triangular pyramid, a rectangular pyramid, a pentagonal pyramid, or a hexagonal pyramid, or a conical shape from which the upper end part 810 is removed, and may include a cavity area that may contain the medicine in the interior thereof and the medicine may be solidified. Then, it may be preferable that the cavity area is located in an upper end area that is above the center of the middle part 820, but according to embodiments, the location, the size, and the form of the cavity area may be variously applied according to a time point, at which the medicine is introduced, an injection time, and a dose of the medical fluid. Moreover, the size and the location of the cavity may be adjusted according to the amount, the vaporization speed, and the temperature of the medicine, the form of the middle part 820 for manufacturing the nano-bubble micro-needle 800, the viscosity of the medicine, the concentration of the medicine, a solvent used, and the thickness of the covering part that covers an upper end of the cavity.

The middle part 820 may be formed of a water-soluble material like the upper end part 810 that penetrates into the skin “S”. However, because the middle part 820 is formed of the compound including the medicine component, it is preferable that the upper end part 810 and the lower end part 830 be formed of different materials.

Then, the medicine component of the middle part 820 may be formed by the bio-compatible material and the additive. For example, the bio-compatible material may include at least any one of carboxy methyl cellulose (CMC), hyaluronic acid (HA), alginic acid, pectin, carrageenan, chondroitin sulfate, dextran sulfate, chitosan, poly lysine, carboxy methyl chitin, fibrin, agarose, pullulan, polyan hydride, poly ortho ester, poly ether ester, poly ester amide, poly butyric acid, poly valeric acid), poly acrylate, ethylene-vinyl acetate polymer, acryl replaced cellulose acetate, poly vinyl chloride, poly vinyl fluoride, poly vinyl imidazole, chloro sulphonate poly olefins, poly ethylene oxide, poly vinyl pirrolidone (PVP), hydroxyl propyl methyl cellulose (HPMC), ethylene cellulose (EC), hydroxyl propyl cellulose (HPC), carboxy methyl cellulose, cyclodextrin, maltose, lactose, trehalose, cellobiose, isomaltose, turanose, and lactulose, or may include at least any one of a copolymer of monomers forming polymers, and cellulose.

Furthermore, the additive may include at least any one of trehalose, oligosaccharide, sucrose, maltose, lactose, cellobiose, hyaluronic acid, alginic acid, pectin, carrageenan, chondroitin sulfate, dextran sulfate, chitosan, poly lysine, collagen, gelatin, carboxy methyl chitin, fibrin, agarose, poly vinyl pirrolidone (PVP), poly ethylene glycol (PEG), poly meta crylate, hydroxyl propyl methyl cellulose (HPMC), ethylene cellulose (EC), hydroxyl propyl cellulose (HPC), carboxy methyl cellulose, cyclodextrin, gentiobiose, alkyl trimethyl ammonium bromide (cetrimide), hexa decyltrimethyl ammonium bromide (CTAB), gentian violet, benzethonium chloride, docusate sodium salt, a SPAN-type surfactant, polysorbate (Tween), sodium dodecyl sulfate (SDS), benzalkonium chloride, and glyceryl oleate.

Further, the medicine component of the middle part 820 may be formed by mixing the bio-compatible material and the effective component. The effective component includes, without being limited thereto, a protein/peptide medicine, and includes at least one of a hormone, a hormone agonist, an enzyme, an enzyme inhibitor, a signal transduction protein or a portion thereof, an antibody and a portion thereof, a single chain antibody, a binding protein or a binding domain thereof, an antigen, an adherence protein, a structural protein, a regulatory protein, a toxo protein, cytokine, a transcriptional regulatory factor, a blood coagulation factor, and a vaccine. In more detail, the protein/peptide medicine may include any one of insulin, insulinlikegrowth factor 1 (IGF-1), a growth hormone, erithropoietin, granulocyte-colony stimulating factors (G-CSFs), granulocyte/macrophage-colony stimulating factors (GM-CSFs), interferon-alpha, interferon-beta, interferon-gamma, interleukin-1 alpha and beta, interleukin-3, interleukin-4, interleukin-6, interleukin-2, epidermal growth factors (EGFs), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRHII), gonadorelin, goserelin, histrelin, leuprorelin, lypressin, octreotide, oxytocin, pitressin, secretin, sincalide, terlipressin, thymopentin, thymosine, triptorelin, bivalirudin, carbetocin, cyclosporine, exedine, lanreotide, luteinizing hormonereleasing hormone (LHRH), nafarelin, parathyroid hormone, pramlintide, enfuvirtide (T-20), thymalfasin, and ziconotide.

Further, the solvent of the medicine component of the middle part 820 may solve the bio-compatible material. The solvent may include at least any one of inorganic solvents and organic solvents including DI water, methanol, ethanol, chloroform dibutyl phthalate, dimethyl phthalate, ethyl lactate, glycerin, isopropyl alcohol, lactic acid, and propylene glycol.

According to the nano-bubble micro-needle 800 according to the embodiment of the present invention, a proper amount of the medicine is injected by forming the cavity of a specific area in the interior of the middle part 820 and injecting the medicine in the liquid state, which is contained in the interior of the cavity, into the interior of the skin “S”, and accordingly, the present invention may reinforce the preservation of the medicine, facilitate the penetration of the nano-bubble micro-needle 800 into the interior of the skin, and allow the medicine in the liquid state to be injected.

The lower end part 830 supports the middle part 820, and is formed of a plurality of nano-bubbles. The lower end part 830 has a prism shape such as a triangular prism shape, a rectangular prism shape, a pentagonal prism shape, or a hexagonal prism shape or a cylindrical shape, and includes the plurality of nano-bubbles in the interior of the shape.

The lower end part 830 may have a diameter and a height of a specific size, and may indicate the depth degree, by which the nano-bubble micro-needle 800 penetrates into the interior of the skin “S”. For example, the depth degree, by which the upper end part 810 and the middle part 820 including the medical fluid penetrate into the interior of the skin “S”, may be estimated according to the diameter and the height of the lower end part 830, and the height of the lower end part 830 may be adjusted according to the depth degree, by which the medicine penetrates, based on the kind of the medicine, the state of the medicine fluid, a time point, at which the medicine is injected, an injection time, and a dose. Furthermore, the diameter of the lower end part 830 may be adjusted according to the weights and the sizes of the upper end part 810 and the middle part 820, a degree, by which the medicine is supported, and a time period, for which the lower end part 830 is fused in the interior of the skin “S”.

The lower end part 830 is formed of a fusible material that connects the base 10 and the micro-needle 800, and separates the micro-needle 800 from the base 10. For example, the lower end part 830 may be formed of a water-soluble fusible material and penetrate into the interior of the skin “S” and be rapidly fused, and accordingly, the nano-bubble micro-needle 800 formed on the base 10 may be rapidly separated.

Then, the lower end part 830 may be formed of a water-soluble material like the upper end part 810 and the middle part 820 that penetrate into the skin “S”. However, the lower end part 830 may be formed of, among the soluble materials, a material that is fused more rapidly than the upper end part 810 and the middle part 820. Because the upper end part 810 is for facilitating the punching of the skin more easily, the middle part 820 includes the medicine and is for more efficient injection, and the lower end part 830 is for rapider separation of the nano-bubble micro-needle 800 formed on the base 10 and the depth degree of the nano-bubble micro-needle 800 in to the interior of the skin “S”, the nano-bubble micro-needle 800 according to the embodiment of the present invention includes the upper end part 810, the middle part 820, and the lower end part 830 of a structure of three layers or more formed of the different materials.

That is, the nano-bubbles that form the lower end part 830 according to the embodiment of the present invention are bubbles formed of the above-described fusible material, that is, the water-soluble material, and the sizes and the amount of the nano-bubbles may be adjusted according to the depth degree, the fusion speed, and the kind of the material of the lower end part 830 that penetrates into the interior of the skin “S”. Because the nano-bubble micro-needle 800 according to the embodiment of the present invention includes the lower end part 830 formed of the plurality of nano-bubbles, it may minimize the weight of the nano-bubble micro-needle 800, may increase the fusion speed of the lower end part 830 as the surface area increases due to the nano-bubbles, and may maintain the strength of the lower end part 830.

Moreover, the nano-bubble micro-needle 800 according to the embodiment of the present invention may adjust the fusion speed, at which the nano-bubbles are fused in the interior of the skin “S” by adjusting the sizes and the amount of the plurality of nano-bubbles formed in the lower end part 830. However, the nano-bubble micro-needle 800 according to the embodiment of the present invention may include the upper end part 810 formed of the nano-bubbles and the middle part 820, as well as the lower end part 830.

Because the lower end part 830 according to the embodiment of the present invention functions to support the upper end part 810 and the middle part 120 in the nano-bubble micro-needle 800, it may indicate the depth degree, by which the nano-bubble micro-needle 100 penetrates. As illustrated in FIG. 8, the lower end part 830 has a prism or cylindrical shape and occupies a size and a volume that are smaller than those of the upper end part 810 and the middle part 820, and accordingly, the lower end part 830 shows an effect of minimizing the area, the volume, and the weight of the nano-bubble micro-needle 800, and supporting the nano-bubble micro-needle 800 such that a proper amount of the medicine is injected due to the shape of the proper size, the proper height, and the proper diameter according to the depth degree, by which the nano-bubble micro-needle 800 penetrates into the skin “S”.

As illustrated in FIG. 8, the nano-bubble micro-needle 800 may be formed on the base 10. The base 10 is not provided with the medicine, and may be separated after the nano-bubble micro-needle 800 of the upper end part 810, the middle part 820, and the lower end part 830 penetrate into the skin “S”. For example, the base 10 is provided in the same form as a kind of a patch, and may be adhered to the skin “S”.

Unlike the nano-bubble micro-needle 800 that penetrates into the skin “S”, the base 10 may be formed of a non-aqueous material that is not fused. Accordingly, because the base 10 does not interfere with the penetration force of the nano-bubble micro-needle 800, it may guide supply of the proper amount of the medicine included in the middle part 820.

For example, the base 10 may be formed of at least any one of a group including poly ethylene (PE), poly propylene (PP), poly tetra fluoro ethylene PTFE, poly methyl meta acrylate (PMMA), ethylene vinyl acetate (EVA), poly capro lactone (PCL), poly urethane (PU), poly ethylene terephthalate (PET), poly ethylene grlycol (PEG), poly vinyl alcohol (PVA), poly lactide (PLA), a poly lactide-glycoride (PLGA) copolymer, and poly glycoric acid.

As illustrated in FIG. 8, the nano-bubble micro-needle 800 according to the embodiment of the present invention may reinforce preservation of the medicine, facilitate penetration of the nano-bubble micro-needle 800 into the skin, inject a proper amount of the medicine, and increase the fusion speed due to the increase in the surface area, by forming the middle part 820 formed of the compound including the medicine, the upper end part 810 located at the upper end of the middle part 820 to facilitate the penetration of the nano-bubble micro-needle 800 into the skin S″, and the lower end part 830 that supports the middle part 820 such that they have a structure of a tree shape of three layers or more.

Furthermore, the nano-bubble micro-needle 800 according to the embodiment of the present invention has a structure of a tree shape of three layers or more, and thus may increase the penetration rate (60% or more) of the structure and the absorption rate of the available substance in the skin, by minimizing the penetration resistance due to the elasticity of the skin when the nano-bubble micro-needle 800 is attached to the skin. Furthermore, the nano-bubble micro-needle 800 of the tree shape can easily penetrate into the skin by applying the structure of three layers or more to maximize the mechanical strength of the structure.

Furthermore, the upper end part 810 of a pyramid or conical shape, the middle part 820, and the lower end part 830 of a prism or cylindrical shape, which form the nano-bubble micro-needle 800 according to the embodiment of the present invention are manufactured through a 3D printing technology. Because the present invention uses a 3D printing method, attachment time may be made very short, precision may also be made high, the price may be made low, the density of the nano-bubble micro-needle 800 in the micro-patch may be increased, and the aspect ratio may be improved as compared with the existing method.

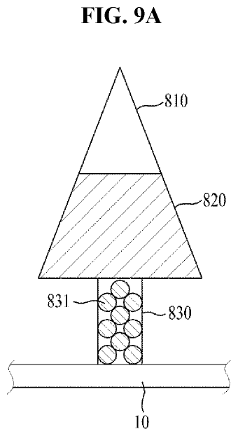

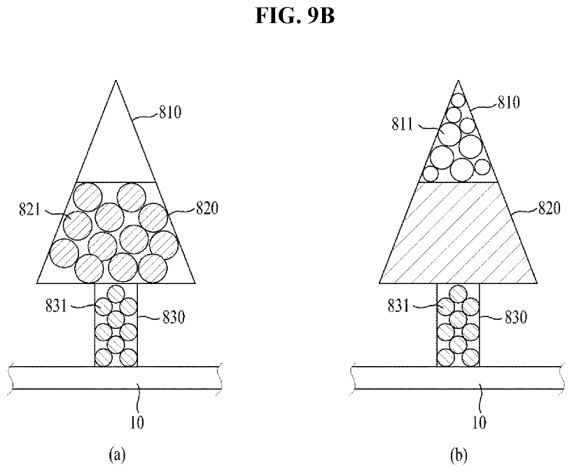

FIGS. 9A and 9B illustrate cross-sectional views of a micro-needle formed of nano-bubbles according to an embodiment of the present invention.

Referring to FIG. 9A, the nano-bubble micro-needle 800 according to the embodiment of the present invention may be formed of the lower end part 830 formed of the plurality of nano bubbles 831, the middle part 820, and the upper end part 810.

The nano-bubble micro-needle 800 according to the embodiment of the present invention includes the lower end part 830 formed of the plurality of nano-bubbles, and as illustrated in FIG. 9B, at least any one of the upper end part 810 and the middle part 820 as well as the lower end part 830 may also be formed of the plurality of nano-bubbles 811 and 821.

The upper end part 810 of the nano-bubble micro-needle 800 may be formed of a plurality of upper end nano-bubbles 811, and the middle part 820 may be formed of a plurality of middle nano-bubbles 821, and the lower end part 830 may be formed of a plurality of lower end nano-bubbles 831. Then, the plurality of nano-bubbles 811, 821, and 831 that form the nano-bubble micro-needle 800 may be formed of the soluble materials that form the upper end part 810, the middle part 820, and the lower end part 830.

Then, the upper end part 810, the middle part 820, and the lower end part 830 that form the nano-bubble micro-needle 800 according to the embodiment of the present invention may be formed of the soluble materials that penetrate into the skin “S” to be fused, and the upper end 810 may be formed of, among the water-soluble materials, a material of a strength that is higher than those of the middle part 820 and the lower end part 830 to facilitate the punching of the skin “S”, and the lower end part 830 may be formed of a material that is fused more rapidly than the upper end part 810 and the middle part 820.

Furthermore, because the middle part 820 is formed of a compound including the medicine component, the middle nano-bubbles 821 may be formed of a compound including a medicine component that is different from those of the upper end nano-bubble 811 and the lower end nano-bubble 831.

For example, the water-soluble material may be at least any one of trehalose, oligosaccharide, sucrose, maltose, lactose, cellobiose, hyaluronic acid, alginic acid, pectin, carrageenan, chondroitin sulfate, dextran sulfate, chitosan, poly lysine, collagen, gelatin, carboxy methyl chitin, fibrin, agarose, poly vinyl pirrolidone (PVP), poly ethylene glycol (PEG), poly meta crylate, hydroxyl propyl methyl cellulose (HPMC), ethylene cellulose (EC), hydroxyl propyl cellulose (HPC), carboxy methyl cellulose, cyclodextrin, and gentiobiose.

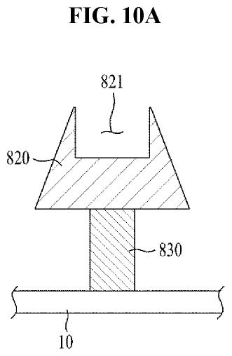

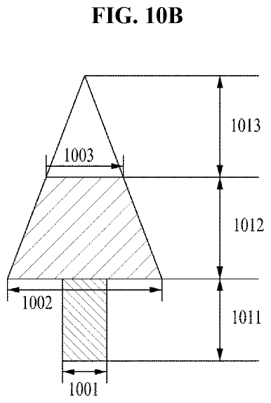

FIGS. 10A and 10B illustrate cross-sectional views for explaining a structural feature of a nano-bubble micro-needle formed according to an embodiment of the present invention.

In detail, FIG. 10A illustrates the cross-sectional view of the nano-bubble micro-needle including the cavity according to another embodiment of the present invention. FIG. 10B illustrates a cross-sectional view of a nano-bubble micro-needle of a structure of three layers or more according to an embodiment of the present invention.

The nano-bubble micro-needle 800 according to another embodiment of the present invention basically includes the middle part 820 formed of the compound including the medicine component, that is, a material that may be solidified, but may include the middle part 820, in which the cavity 122 is formed, such that the middle part 820 includes the medicine in the liquid state according to applied embodiments. According to the embodiment, as illustrated in FIG. 9B, when the middle part 820 is formed of the plurality of nano-bubbles 821, the plurality of nano-bubbles 821 may be formed in the interior of the middle part 820, in which the cavity 822 is formed.

Referring to FIG. 10A, the nano-bubble micro-needle 800 according the another embodiment of the present invention may include the middle part 820 including the cavity 822. The cavity 122 may have a groove shape in the middle part 820, and may have a form and a size for including the medicine.

Then, the surface of the cavity in contact with the medicine may be coated with the waterproof material. When the nano-bubble micro-needle 800 according the another embodiment of the present invention may include the cavity 822, it may include the medicine in the liquid state. Accordingly, because the medical fluid in the liquid state may be absorbed by the middle part 820, the surface of the cavity is coated with the waterproof material to block the medical fluid.

For example, the surface of the cavity may be coated with the waterproof material including a mineral-based material or a lipid-based material. Here, the waterproof material may include at least one of beeswax, oleicacid, soy fatty acid, castor oil, phosphatidylcholine, d-α-tocopherol/Vitamin E, corn oil, corn oil mono-ditridiglycerides, Cottonseed oil, olive oil, peanut oil, peppermint oil, safflower oil, sesame oil, soybean oil, hydrogenated vegetable oils, hydrogenated soybean oil, caprylic/capric triglycerides derived from coconut oil or palm see oil, and phosphatidylcholine, and a mixture thereof.

According to the embodiment, the surface of the cavity may be coated with different waterproof agents according to the kind and the state of the medicine injected into the cavity 822, and the size, the height, and the form of the cavity 822 may be formed in the middle part 820 in different shapes according to the kind of the medicine, the state of the medicine, the time point, at which the medicine is injected, the injection time period, and the dose.

Referring to FIG. 10B, the nano-bubble micro-needle 800 according to the embodiment of the present invention is a micro-structure having three layers or more, and includes the upper end part 810 and the middle part 820 of a pyramid or conical shape, and the lower end part 830 of a prism or cylindrical shape.

As illustrated in FIG. 10B, the diameter 1002 of the bottom surface of the middle part is larger than the diameter 1003 of the bottom surface of the upper end part or the diameter 1001 of the bottom surface of the lower end part, and the diameter 1003 of the bottom surface of the upper end part is larger than the diameter 1001 of the bottom surface of the lower end part. Their sizes may be determined in the sequence of the diameter 1002 of the bottom surface of the middle part, the diameter 1003 of the bottom surface of the upper end part, and the diameter 1001 of the bottom surface of the lower end part.

Furthermore, the height 1012 of the middle part may be larger than the height 1013 of the upper end part, and the sum of the height 1012 of the middle part and the height 1013 of the upper end part may be larger or smaller than the height 1011 of the lower end part. That is, in the nano-bubble micro-needle 800 according to the embodiment of the present invention, the height 1012 of the middle part may be largest, and the height 1013 of the upper end part and the height 1011 of the lower end part may be the same or may vary according to the embodiment, to which the nano-bubble micro-needle 800 according to the embodiment of the present invention is applied. However, the height 1011 of the lower end part, the height 1012 of the middle part, and the height 1013 of the upper end part of the nano-bubble micro-needle 800 according to the embodiment of the present invention are not limited to those illustrated in FIG. 10B, and may be various heights according to the embodiment applied.

Because the middle part 820 of the nano-bubble micro-needle according to the embodiment of the present invention has the cavity containing the medicine, it may have the widest volume, the largest bottom surface diameter 1002, and the largest height 1012. Because the upper end part 810 has a pyramid or conical shape for penetrating into the skin “S”, the diameter 1003 of the bottom surface of the upper end part is the same as the diameter of the upper surface (or the tip end) of the middle part, and may be determined according to the cross-sectional area of the pyramid or conical shape that forms the middle part 820. Furthermore, the height 1013 of the upper end part may be determined according to the pyramid or conical shape of the middle part.

Because the lower end part 830 of the nano-bubble micro-needle according to the embodiment of the present invention functions to support the upper end part 810 and the middle part 820 in the nano-bubble micro-needle 800, it may indicate the depth degree, by which the nano-bubble micro-needle 800 penetrates into the skin. Accordingly, the lower end part 830 has a volume and a bottom surface diameter 1001 that are smaller than those of the upper end part 810 and the middle part 820. However, the height 1011 of the lower end part may be determined according to the depth degree, by which the nano-bubble micro-needle penetrates into the skin.

The lower end part 830 has a prism or cylindrical shape and includes the bottom surface diameter 1003 that is smaller than the diameter 1002 of the bottom surface of the upper end part and the diameter 1001 of the bottom surface of the middle part, and the volume of the lower end part 130 is also smaller than those of the upper end part 810 and the middle part 820. The lower end part 830 is adapted to indicate the depth degree, by which the nano-bubble micro-needle 800 penetrates into the skin “S” and supports the upper end part 810 and the middle part 820, and thus minimizes the area, the volume, and the weight of the nano-bubble micro-needle 100 according to the embodiment of the present invention. Accordingly, the lower end part 830 shows an effect of supporting the nano-bubble micro-needle 800 that penetrates into the interior of the skin “S” such that a proper amount of the medical fluid may be injected due to a proper size, a proper height, and a proper diameter shape according to the depth degree of the nano-bubble micro-needle 800.

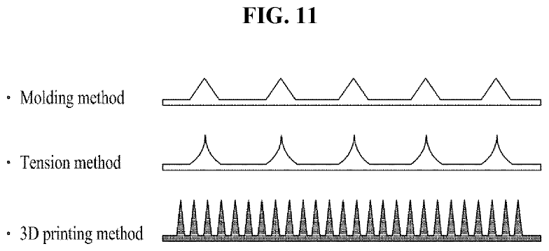

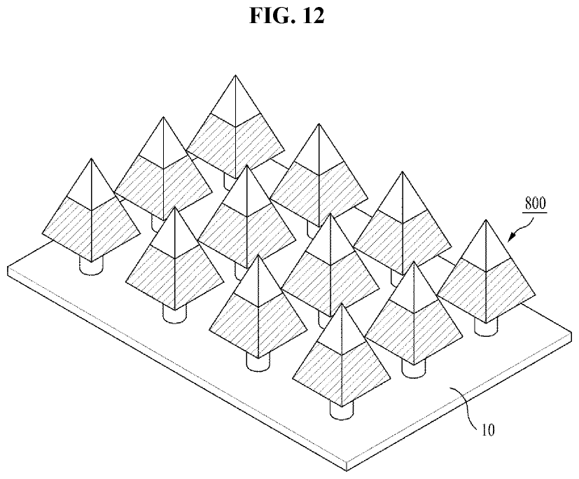

FIG. 11 illustrates exemplary views for comparing micro-needles manufactured according to an existing method and a method of the present invention. FIG. 12 illustrates a perspective view of a nano-bubble micro-needle patch manufactured according to an embodiment of the present invention.

Referring to FIG. 11, it can be seen that the density of the micro-needles is low in the molding method and the tension method whereas the density of the nano-bubble micro-needles according to the embodiment of the present invention manufactured by using a stacking method, for example, a 3D printing method is very high as compared with the existing methods due to the limitation of the molding method and the tension method, and it can be seen that the aspect ratio of the nano-bubble micro needles manufactured by the method according to the present invention is also higher than those of the molding method and the tension method is very high. Of course, the aspect ratio of the nano-bubble micro-needle may be adjusted in the method according to the present invention, and the aspect ratio may be determined according to a field, in which the nano-bubble micro-needle of the present invention is used, for example, a treatment or medical field.

According to the method (the 3D printing method) according to the present invention, it is advantageous to punch skin, there is no pain, and the density of the nano-bubble micro needles is higher than those of the molding method and the tension method. Furthermore, it can be seen that attachment time is very short as compared with the existing method in the method according to the present invention, it can be seen that precision is also high, and it can be seen that manufacturing costs are low and thus expansion performance is high as it uses a stacking method, for example, the 3D printing method. In this way, the method according to the present invention is very advantageous in the technical aspect and the economical aspect as compared with the existing methods, such as the molding method and the tension method.

That is, in the nano-bubble micro-needle realized by the stacking technology through the method according to the present invention, skin may be punched well and pains are very low due to the high aspect ratio and the attachment time is very short due to the high density of the nano-bubble micro-needles. In addition, the present invention may realize the nano-bubble micro-needle at a high precision of about 5 micrometers and a desired medicine may be disposed at a desired location, and thus the expansion performance is high.

The nano-bubble micro-needle 800 manufactured as described above, as illustrated in FIG. 12, may be manufactured by a plurality of nano-bubble micro-needle patch formed on the base 10, and may be easily applied to the medical field. That is, the present invention may secure a high competition in the medical market field by manufacturing the nano-bubble micro-needle 800 of the structure of three layers or more in the stacking method using the 3D printing.

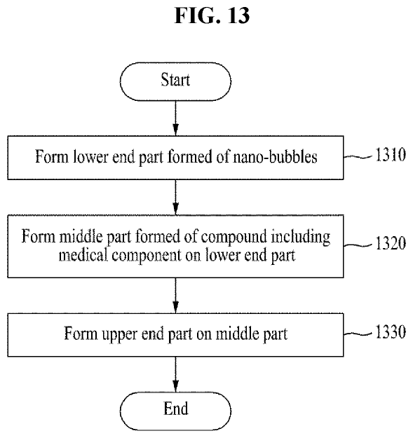

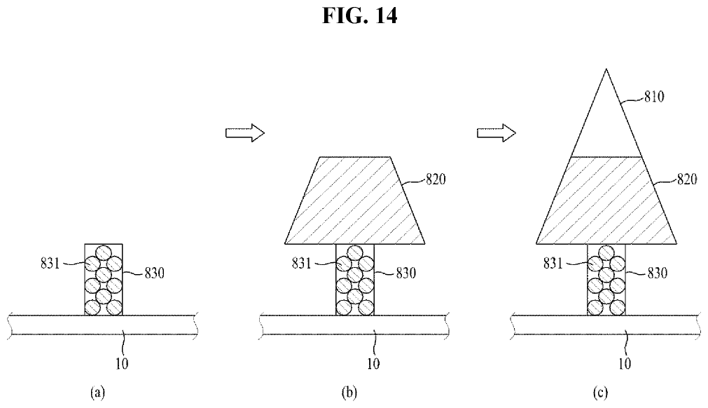

FIG. 13 illustrates a flowchart of operations of a method for manufacturing a nano-bubble micro-needle according to an embodiment of the present invention. FIG. 14 illustrates operations of manufacturing a micro-needle through a nano-bubble method for manufacturing a nano-bubble micro-needle according to an embodiment of the present invention.

The nano-bubble micro-needle 800 according to the present invention illustrated in FIG. 14, which is manufactured through the manufacturing method of FIG. 13 is manufactured through the 3D printing method.

Referring to FIGS. 13 and 14A, in operation 1310, the lower end part 830 formed of nano-bubbles is formed. In the method for manufacturing the nano-bubble micro-needle according to the embodiment of the present invention, the lower end part 830 having the plurality of nano-bubbles 831 in the interior of the prism or cylindrical shape may be formed on the base 10.

The lower end part 830 may have a diameter and a height of a specific size, and may indicate the depth degree, by which the nano-bubble micro-needle 800 penetrates into the interior of the skin “S”. For example, the depth degree, by which the upper end part 810 and the middle part 820 including the medical fluid penetrate into the interior of the skin “S”, may be estimated according to the diameter and the height of the lower end part 830, and the height of the lower end part 830 may be adjusted according to the depth degree, by which the medicine penetrates, based on the kind of the medicine, the state of the medicine fluid, a time point, at which the medicine is injected, an injection time, and a dose. Furthermore, the diameter of the lower end part 830 may be adjusted according to the weights and the sizes of the upper end part 810 and the middle part 820, a degree, by which the medicine is supported, and a time period, for which the lower end part 830 is fused in the interior of the skin “S”.

Furthermore, the lower end part 830 is formed of a fusible material that connects the base 10 and the micro-needle 800, and separates the micro-needle 800 from the base 10. For example, the lower end part 830 may be formed of a water-soluble fusible material and penetrate into the interior of the skin “S” and be rapidly fused, and accordingly, the nano-bubble micro-needle 800 formed on the base 10 may be rapidly separated.

Then, the lower end part 810 may be formed of a water-soluble material like the upper end part 810 and the middle part 820 that penetrate into the skin “S”. However, the lower end part 830 may be formed of, among the soluble materials, a material that is fused more rapidly than the upper end part 810 and the middle part 820. Because the upper end part 810 is for facilitating the punching of the skin more easily, the middle part 820 includes the medicine and is for more efficient injection, and the lower end part 830 is for rapider separation of the micro-needle 800 formed on the base 10 and the depth degree of the nano-bubble micro-needle 100 in to the interior of the skin “S”, the nano-bubble micro-needle 800 according to the embodiment of the present invention includes the upper end part 810, the middle part 820, and the lower end part 830 of a structure of three layers or more formed of the different materials.

That is, the nano-bubbles that form the lower end part 830 according to the embodiment of the present invention are bubbles formed of the above-described fusible material, that is, the water-soluble material, and the sizes and the amount of the nano-bubbles may be adjusted according to the depth degree, the fusion speed, and the kind of the material of the lower end part 830 that penetrates into the interior of the skin “S”. Because the nano-bubble micro-needle 800 according to the embodiment of the present invention includes the lower end part 830 formed of the plurality of bubbles, it may minimize the weight of the nano-bubble micro-needle 800, may increase the fusion speed of the lower end part 830 as the surface area increases due to the nano-bubbles, and may maintain the strength of the lower end part 830.

In operation 1320, the middle part 820 formed of a compound penetrating into an interior of skin and including the medicine component is formed on the lower end part 830. As illustrated in FIG. 14B, in the method for manufacturing the nano-bubble micro-needle according to the embodiment of the present invention, the solidified middle part 820 formed of the compound including the medicine component may be formed on the lower end part 830. However, although FIG. 14B illustrates the middle part 820 formed of the compound including the medicine component, the middle part 820 of the nano-bubble micro-needle 800 according to another embodiment of the present invention may include the cavity that may include the medicine in the liquid state.

Then, the middle part 810 may be formed of a water-soluble material like the upper end part 810 that penetrates into the skin “S”. However, because the middle part 820 is formed of the compound including the medicine component, it is preferable that the upper end part 810 and the lower end part 830 be formed of different materials.

In operation 1330, the upper end part 810 is formed on the middle part 820. Referring to FIG. 14C, in the method for manufacturing the nano-bubble micro-needle according to the embodiment of the present invention, the upper end part 810 that facilitates the penetration of the nano-bubble micro-needle into the skin “S” may be formed to be located at the upper end of the middle part 820. A tip end of the upper end part 810 has a sharp end shape with respect to the penetration direction, in which the nano-bubble micro-needle 800 penetrates into the skin “S”, and may have, for example, a pyramid or conical shape to facilitate the penetration of the nano-bubble micro-needle 800 into the skin “S”.

The upper end part 810, the middle part 820, and the lower end part 830 of the nano-bubble micro-needle 800 according to the embodiment of the present invention may be formed of different materials. The upper end part 810, the middle part 820, and the lower end part 830 may be formed of the same water-soluble material, but the upper end part 810 that facilitates the penetration, the middle part 820 formed of the compound including the medicine component, and the lower end part 830 that supports the middle part 820 and facilitates the separation from the base 10 may be formed of materials of different characters as long as they are soluble materials.

Although the embodiments of the present invention have been described with reference to the limited embodiments and the drawings, the present invention may be variously corrected and modified from the above description by those skilled in the art to which the present invention pertains. For example, the above-described technologies can achieve a suitable result even though they are performed in different sequences from those of the above-mentioned method and/or coupled or combined in different forms from the method in which the constituent elements such as the system, the architecture, the device, or the circuit are described, or replaced or substituted by other constituent elements or equivalents.

Therefore, the other implementations, other embodiments, and the equivalents of the claims pertain to the scope of the claims.

### INDUSTRIAL APPLICABILITY

1. A liquid injection type micro-needle having a structure of three layers or more, the liquid injection type micro-needle comprising:
a middle part penetrating into an interior of skin and including a fusible medical fluid in a cavity thereof;
a lower end part supporting the middle part; and
an upper end part located at an upper end of the middle part and configured to facilitate the penetration.

2. The liquid injection type micro-needle of claim 1, wherein the middle part includes the cavity having a groove shape of a specific size in an interior thereof, and the medical fluid in a liquid state is contained in the cavity.

3. The liquid injection type micro-needle of claim 2, wherein the middle part seals the medical fluid by blocking an upper end of the cavity, in which the medical fluid is contained.

4. The liquid injection type micro-needle of claim 2, wherein a surface of the cavity, which contacts the medical fluid, is coated with a waterproof material, which does not react with the medical fluid.

5. The liquid injection type micro-needle of claim 1, wherein the upper end part and the middle part have a pyramid or conical shape, and the lower end part has a prism or cylindrical shape.

6. The liquid injection type micro-needle of claim 1, wherein the lower end part includes a fusible material connecting a base and the liquid injection type micro-needle, and separates the liquid injection type micro-needle from the base.

7. The liquid injection type micro-needle of claim 1, wherein the upper end part, the middle part, and the lower end part are formed of different materials.

8. (canceled)

9. A nano-bubble micro-needle having a structure of three layers or more, the nano-bubble micro-needle comprising:
a middle part formed of a compound penetrating an interior of skin and including a medicine component;
a lower end part supporting the middle part and formed of a plurality of nano-bubbles; and
an upper end part located at an upper end of the middle part and configured to facilitate the penetration.

10. The nano-bubble micro-needle of claim 9, wherein the upper end part and the middle part have a pyramid or conical shape, and the lower end part has a prism or cylindrical shape.

11. The nano-bubble micro-needle of claim 10, wherein the upper end part, the middle part, and the lower end part are formed of different materials.

12. The nano-bubble micro-needle of claim 11, wherein the lower end part includes the plurality of nano-bubbles in an interior of the prism or cylindrical shape.

13. The nano-bubble micro-needle of claim 12, wherein sizes and the amount of the nano-bubbles are adjusted according to a depth degree, a fusion speed, and a kind of a material of the lower end part penetrating into the skin.

14. The nano-bubble micro-needle of claim 9, wherein the lower end part is formed of a fusible material connecting a base and the nano-bubble micro-needle, and separates the nano-bubble micro-needle from the base.

15. (canceled)
